(19) United States
(12) Patent Application Publication
(10) Pub. No.: US 2026/0054041 A1
(43) Pub. Date: Feb. 26, 2026

(54) METHODS AND APPARATUS FOR PREVENTING CLOGGING OF IMPLANTABLE SHUNTS AND THE LIKE

(71) Applicant: The Regents of the University of California, Oakland, CA (US)

(72) Inventors: James Friend, San Diego, CA (US); David Santiago-Dieppa, San Diego, CA (US); Aditya Vasan, San Diego, CA (US); Yau C. Yun, San Diego, CA (US)

(21) Appl. No.: 19/305,558

(22) Filed: Aug. 20, 2025

Related U.S. Application Data
(60) Provisional application No. 63/685,581, filed on Aug. 21, 2024.

(51) Int. Cl. A61M 27/00; A61M 25/00; A61M 25/10

(52) U.S. Cl. CPC A61M 27/006; A61M 25/10182; A61M 2025/0001; A61M 2025/109; A61M 2205/3592; A61M 2209/04

(57) ABSTRACT

The invention relates to methods and apparatus for unclogging implantable shunts, pored catheters, and other medically implantable lumen with openings for infiltration of bodily fluids and the like.

### RELATED APPLICATIONS

This application claims priority to U.S. provisional patent application No. 63/685,581 filed on Aug. 21, 2024, titled PREVENTING CLOGGING OF VENTRICULOPERITONEAL SHUNTS FOR HYDROCEPHALUS, which is incorporated by reference herein in its entirety.

### FIELD OF THE INVENTION

The invention relates to methods and apparatus for unclogging implantable shunts, pored catheters, or other medically implantable lumen with openings for infiltration of bodily fluids and the like.

### BACKGROUND

Implantable shunt systems for transporting bodily fluids from one place in the body to another (or external of the body) are well known in the medical field. Such systems typically comprise a catheter implanted within a bodily cavity. The catheter typically has holes to allow infiltration of a bodily fluid that needs to be drained from that bodily cavity into the inner lumen of the catheter. The inner lumen of the catheter typically is further in fluid communication with a tube to transport the fluid away from the bodily cavity/catheter to a safer place in the body or external of the body.

Hydrocephalus is a neurological disorder in which there is an excess accumulation of cerebrospinal fluid (CSF) within the intracranial compartment, often resulting from the obstruction of CSF flow. The accumulation of CSF, an incompressible Newtonian fluid,¹ can enlarge the four interconnected ventricular cavities—a pair of lateral ventricles and the third and fourth ventricle. The CSF usually flows via a cerebral aqueduct between the third and fourth ventricles.² Secreted from the choroid plexus (ChP) and stored in the ventricular system,³ the CSF has a total volume in the human body of 150 mL, with 40 mL of it within the four ventricles of the brain.^{4,5} The ChP is a layer of epithelial tissue located on the ventricle wall with a variety of transport proteins that regulate the secretion of CSF.^{6,7} The CSF supports several key physiological functions including the maintenance of ionic homeostasis, shock absorption, and the integrity of the blood-brain barrier.⁷⁻¹⁵ The CSF is composed of proteins, albumin, immunoglobulins, vitamins, lipids, and ions important in neural function.^{10,16}.

Patients with hydrocephalus often experience headaches, poor body coordination, an increase in sleepiness, blurred vision, hearing loss, and even loss of bladder control.^{17,18} There are numerous causes of hydrocephalus; these include infections;¹⁹ complications in surgery, tumors, and disease;^{20,21} aqueductal stenosis in infants;²² and the abnormal production of CSF from subarachnoid hemorrhage and head trauma.²³ For pediatric patients, untreated hydrocephalus can hinder mental development and produce learning disabilities.^{24,25} Current treatment methods include buccal medication such as acetazolamide^{26,27}, neurosurgery, and implants.

The current standard for treating hydrocephalus is ventricular peritoneal shunting (VPS). About 36,000 shunts are implanted each year.²⁸ Some shunts include a programmable valve to regulate the CSF drainage at greater expense,²⁹ while others integrate flow and pressure sensors³⁰ or an external magnet-adjustable valve to adjust the flow post-implantation.³¹

In VPS, a catheter is surgically implanted in the brain. The catheter has holes (or pores) along its surface through which CSF can flow into the catheter. The catheter is connected to a lumen that extends down into the abdomen, where the excess CFS can drain harmlessly out of the shunt system into the patient's stomach. Positioned in the lumen between the brain catheter and the abdomen is a resistor, comprising a reservoir for receiving the CFS followed by a limiter valve. The pressure limiter valve is configured to open to allow CFS to flow down into the abdomen when the upside pressure in the catheter exceeds a preset threshold, that preset threshold being set to a value indicative of above-normal pressure in the brain. The pressure at the limiter valve in the catheter is normally indicative of the pressure in the brain and above-normal pressure in the brain is indicative of excessive CFS in the brain (i.e., hydrocephalus).

Another method is endoscopic third ventriculostomy (ETV). It is a minimally invasive procedure to restore the homeostasis of CSF by creating an alternative circulation pathway in the third ventricle to the subarachnoid artery.³² During an ETV, the surgeon punctures the floor of the third ventricle, creating an alternative pathway for CSF circulation.^{32,33} This procedure often goes hand in hand with the installation of a ventriculoperitoneal (VP) shunt when there is an infection or malfunction of the previous VP shunt,³⁴ or may be a preferred option for patients with obstructive hydrocephalus.³⁵ In most new cases, the installation of a VP shunt is preferred over an ETV, though an ETV is an appropriate alternative to revision of a malfunctioning shunt.³⁶

Unfortunately, shunts are unreliable and can produce under-drainage or over-drainage of CSF.³⁷ Under-drainage typically occurs from obstruction of the shunt, requiring revision surgery: in a sample size of 227 patients with 90 shunt revisions performed in 2021, 30% were related to obstruction of the catheter.³⁸ In the United States in 2005, the last year complete data is available, one-half of the $1B annual expenditures in shunt procedures is due to revision surgeries.³⁹ In the United Kingdom, the reported cumulative shunt revision rate is 24% for children and 14% for adults in the first year after installation.⁴⁰ Gupta, et al., found that 54% of patients with shunts require at least four shunt revisions and that some patients also experience shunt infections.⁴¹ The infection rate ranges from 5% to 15% within the first month after shunt installation surgery.⁴² A Danish study considered two groups of patients with shunts implanted approximately 50 years apart and found no difference in their revision rates,⁴³ implying that, regardless of advancements in care and practice, there were no significant changes in treatment outcomes. Improvements in the existing shunt system are clearly needed. In particular, there is a pressing need to address shunt obstruction as one of the most common causes of shunt revision.⁴⁴ The obstruction of the shunt often results from blockage of the draining ports by proteins and coagula⁴⁵ and rarely the growth of the choroid plexus into the shunt.³⁷

Shunt obstructions are considered a medical emergency⁴⁶ and can rapidly lead to serious con-sequences, particularly an increase in intracranial pressure. This can trigger altered mental states, bradycardia, vomiting, infection, or other symptoms associated with hydrocephalus. Pharmacological and surgical intervention is necessary and typically requires hospitalization. A common intervention is a retrograde flush into the shunt that is used to remove the occlusion.⁴⁰ Newer shunts employ a combination of silicone and nitinol instead of plastic;²² these shunts can change their size due to the property of nitinol wire while the silicone controls the initial geometry of the shunt.^{47,48}

Recent efforts to improve VP shunts have used passive and active methods in an attempt to reduce or clear the clogging responsible for a majority of the revision surgeries and emergency room visits for hydrocephalus patients. Galarza, et al., sought to optimize the size and location of the perforations in the shunt as a passive method to reduce the risk of clogging. They employed finite element analysis and optimization to design an improved shunt.⁴⁹ While promising, the risk of clogging is generally so significant that a passive means to reduce this risk still leaves one with a shunt that could clog in many patients. Adopting an active approach instead, Patel, et al.,⁵⁰ describe the use of CSF passed into the shunt in a recirculating flow propelled either by a micropump integrated near the proximal end of the shunt, or an electromagnetically-driven diaphragm pump. The downside of this approach is the increased risk of infection due to the passage of the CSF back into the brain from the outside, and, based on the results they present, it is not entirely clear if the approach was successful. Lutz, et al., suggest that introducing sensors and a means to communicate the status of the shunt to a patient's mobile phone or similar could help reduce the risk of shunt clogging and the adverse outcomes when clogs occur. They also note that although the problem is well understood, “smart shunts” as a potential solution were still not available.⁵¹

Persons who have hydrocephalus often require VPS for an extended period of time, frequently for lifetime. Thus, clogging of the pores of the catheter for such persons is far too common. Short-term VPS also is performed frequently in a clinical setting. Short-term VPS in a clinical setting is quite similar to that described above, except that the CFS drains into a container external of the patient's body. Even in the short-term scenario, it is not uncommon for the pores in the catheter to become clogged with metabolites and/or other coagula.

In fact, there are many medical applications for pored catheters in which it is possible for the pores to become clogged, leading to reduced effectiveness of medical treatment, harmful symptoms, or worse. Some such applications include urinary catheters, lumbar peritoneal shunts, PICC (Peripherally Inserted Central Catheter) lines, etc.

### SUMMARY

In a first embodiment, a ventriculoperitoneal (VP) shunt system comprises a catheter for placement in a ventricle, the catheter comprising a tube, the tube having an outer diameter and an inner diameter, and defining an inner lumen, the catheter tube having a plurality of holes in a wall thereof to allow infiltration of fluids from external of the catheter into the inner lumen via the holes, a drainage tube in fluid communication with the inner lumen of the catheter for draining fluid that infiltrates the catheter and an inflatable tube disposed within the inner lumen of the catheter, the inflatable tube having a diameter smaller than a diameter of the inner lumen of the catheter when the inflatable tube is uninflated and inflatable to a diameter greater than the outer diameter of the catheter.

In a second embodiment, a method of unclogging an in vivo artificial tube having an outer diameter and an inner diameter, and defining an inner lumen, the tube having a plurality of holes in a wall thereof to allow infiltration of fluids from external of the tube into the inner lumen via the holes, comprises disposing an inflatable tube within the inner lumen of the artificial tube, the inflatable tube having a diameter smaller than a diameter of the inner lumen when the inflatable tube is uninflated and inflatable to a diameter greater than the outer diameter of the artificial tube, detecting a condition indicative of clogging of the artificial tube, and inflating the inflatable tube sufficiently to contact the wall of the artificial lumen and protrude through the holes.

### BRIEF DESCRIPTION OF THE DRAWINGS

The details of one or more variations of the subject matter described herein are set forth in the accompanying drawings and the description below. Other features and advantages of the subject matter described herein will be apparent from the description and drawings, and from the claims.

The accompanying drawings, which are incorporated in and constitute a part of this specification, show certain aspects of the subject matter disclosed herein and, together with the description, help explain some of the principles associated with the disclosed implementations. In the drawings,

FIG. 1 is a diagram illustrating a VPS system disposed in a human body in accordance with an embodiment;

FIG. 2A is a cut-away plan view of a catheter according to a first implementation;

FIG. 2B is a blow up view of portion 2B of FIG. 2A;

FIG. 2C is a blow up view of portion 2C of FIG. 2A with the balloon tube omitted;

FIG. 2D is a cut-away plan view of the distal end of the catheter system of the first implementation with the inflatable balloon tube uninflated;

FIG. 2E is a cut-away plan view of the proximal end of the catheter system of the first implementation with the inflatable balloon tube inflated;

FIG. 2F is a cross-sectional view of the proximal end of the original catheter of the Codman® VP shunt that was modified to create the first implementation;

FIG. 2G is a cross-sectional view of the proximal end of the catheter of the first implementation after modification;

FIG. 3A is a diagram illustrating a model apparatus for testing the ventricular catheter, intracranial pressure, and CSF flow in accordance with an embodiment;

FIG. 3B is an analogous circuit diagram corresponding to the model of FIG. 3A;

FIG. 4A is graph showing plots of intracranial pressure as a function of time for a catheter not including the inflatable balloon tube with and without the addition of a clogging agent into the ventricle; and

4B is graph showing plots of intracranial pressure as a function of time for a catheter including the inflatable balloon tube with and without the addition of a clogging agent into the ventricle.

### DETAILED DESCRIPTION

In some embodiments, there is provided a next-generation ventriculoperitoneal shunt system configured to safely induce unclogging via internal or external actuation.

In some embodiments, the next-generation ventriculoperitoneal shunt system integrates an expandable silicone tube located within the internal lumen of the ventricular catheter that, when clogging occurs, may be inflated to press against the inner wall of the ventricular catheter and protrude slightly through the pores in the catheter to dislodge and/or break down into smaller pieces any coagula that has built up in the pores, thereby unclogging the pores.

The material and dimensions of the tube should be selected to achieve certain goals. First, the tube should be small enough, such that, when uninflated, its diameter is so small that it does not significantly reduce flow in the ventricular catheter compared to the same catheter with no internal tube. Preferably, the diameter of the tube is less than about 10% of the inner diameter of the ventricular catheter. Second, the tube should have a maximum strain (the amount of expansion possible before rupture) sufficient to allow the tube to expand to slightly larger than the outer diameter of the ventricular catheter (so that the tube will protrude from the pores when inflated). Third, the tube should be inflatable to expand to the desired diameter under a fairly low pressure (e.g., a pressure that is safe within the human body in the case of rupture of the tube). Fourth, the material of the tube should be biocompatible. Fifth, the inflatable tube material should be soft enough to allow the portions of the tube adjacent to the pores in the catheter to protrude out of those pores when inflated.

In an embodiment, the silicone forming the tube may have a maximum uniaxial strain of about 900%, making it possible to dispose the tube into the ventricular catheter's inner lumen without significant reduction in the lumen's free cross-sectional area. Strain is a measurement of the expandability of a material and is usually given as a percentage. A uniaxial strain of 900% thus means that the length of the material may increase by 900% while loaded along a single direction without failure. In the present example, the pressure-driven loading is biaxial, both along the length of the tube and around its circumference. A maximum uniaxial strain of 900% converts to a maximum biaxial strain of 608% via equivalent principal stretches for the uniaxial and biaxial strain cases:

$${I}_{1}^{b i} = 2 {λ}_{b}^{2} + {λ}_{b}^{- 4} = {I}_{1}^{u n i} = {λ}_{u}^{2} + 2 {λ}_{u}^{- 1}$$

where λᵤ and λ_{b} are the uniaxial and biaxial stretches, respectively, and the stretch is one plus the strain value. A maximum biaxial strain of 608% implies that the diameter of the tube can be increased by 608% without failure. With a safety factor of 2, this implies a maximum diameter change of 304%.

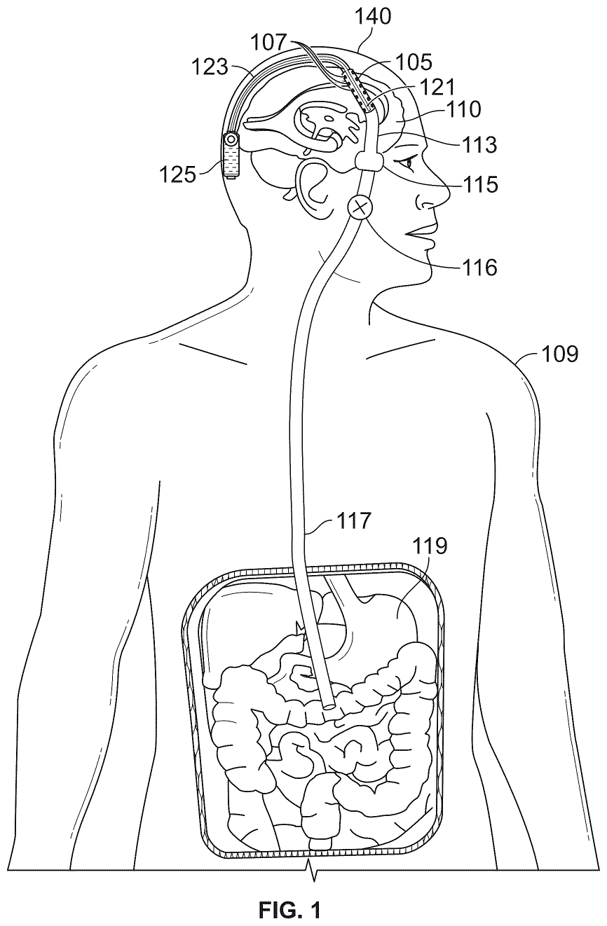

FIG. 1 is a diagram illustrating a VPS system disposed in a human body in accordance with one embodiment. FIG. 1 is intended to demonstrate the basic components of the system in vivo in an easily perceptible manner and at a conceptual level and, thus, is not to scale and is partially schematic. The system includes a ventricular catheter 105 positioned in the brain 110 of the patient 109. The catheter includes a number of pores 107 in its wall through which CSF in the brain may infiltrate the inner lumen of the catheter 105. The inner lumen of the catheter is connected by a tube 113 to a small reservoir 115 at the proximal side of a limiter valve 116. The limiter valve 116 is configures so that it will open and allow fluid to flow from the proximal side to the distal side of the valve when the pressure on the proximal side of the valve exceeds the pressure on the distal side of the valve by a predetermined threshold. That threshold will be set to a pressure that slightly below a pressure that would be indicative of hydrocephalus in the brain. That pressure may vary for different patients, but, generally, is between about 6-25 cmH₂O in adults⁶⁸ and is lower in children and infants⁷¹.

The distal end of the valve is connected to another tube 117 that runs down into the abdomen 119, where the CSF that flows into the catheter 105 through the pores 107, into the reservoir 115, and through the valve 116, can drain, thus keeping the patient from experiencing hydrocephalus.

An expandable balloon tube 121 is disposed coaxially within the lumen of the ventricular catheter 105. The balloon 121 is connected via another tube 123 that is generally not expandable to another reservoir 125 located close to the scalp 140 of the patient 109. This reservoir 125 may be filled with water, saline, or another biocompatible fluid.

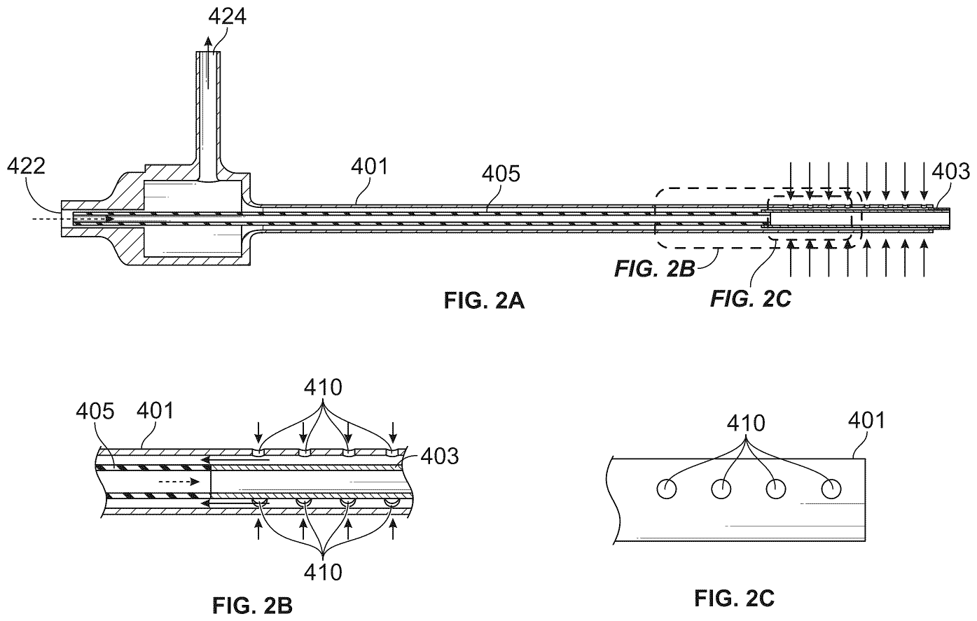

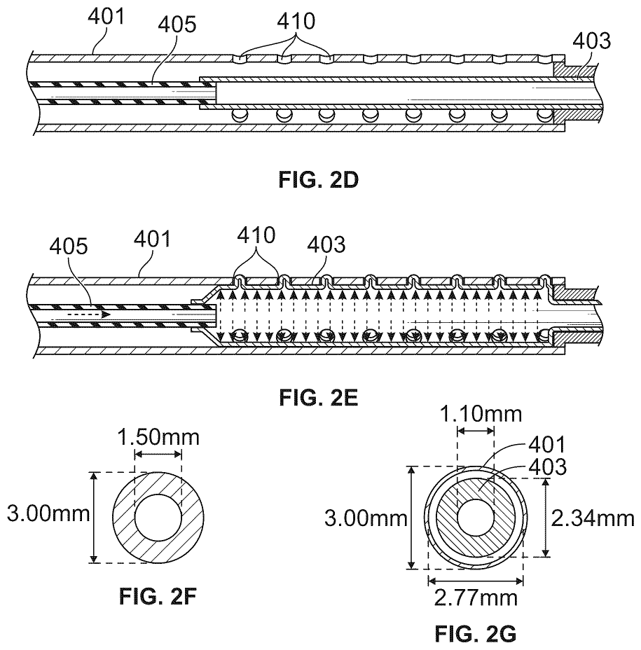

The reservoir 125 is made of a material that easily deforms under pressure such that, when compressed (pressurized), the fluid will flow out of the reservoir 125, through the tube 123, and into the balloon 121 to cause the balloon to inflate. The reservoir 125 contains sufficient fluid to inflate the balloon tube so that the diameter of the balloon, when filled with the water from the reservoir 125 would cause the balloon to expand to an unrestricted diameter slightly greater than the outer diameter of the ventricular catheter, such as shown in FIG. 2E. Under these circumstances, the wall of the ventricular catheter 105 will prevent most of the balloon tube 121 from expanding to a diameter greater than the inner diameter of the ventricular catheter over most of the surface area of the balloon. However, where the surface of the balloon 121 is coincident with the pores 107 of the catheter 105, the balloon tube 121 will protrude from such pores. Thus, inflating the balloon tube 121 to an unrestricted diameter slightly greater than the outer diameter of the catheter 105 will force any material that might be clogging the pores 107 out of the pores, thereby unclogging the catheter 105. Note that the force applied to the edges of the pores by the inflated balloon tube 121 should dislodge any material clogging the pores and/or break it up into smaller pieces. Most of the dislodged or broken up material will (1) travel to other locations in the brain where they do not interfere with the operation of the VPS system, (2) be resorbed into the body, or (3) pass more easily into the catheter 105 without causing clogging, through the valve 116, and into the abdomen 119 where it can eventually be eliminated from the body.

As the inflated balloon tube 121 will also press against the inner wall of the catheter 105, it may also dislodge, break up, or compress any material that has formed on the inner wall of the catheter, thereby also improving flow characteristics inside the catheter.

In one embodiment, the reservoir 125 is implanted sufficiently close to the scalp 140 that an individual, e.g., the patient him/herself, can press against their scalp above the reservoir to compress it to cause the fluid contained therein to flow out of the reservoir 125, through the tube 123, and into the balloon tube 121, thereby inflating the balloon tube.

In other embodiments, the reservoir may include electronics (including, for instance, a radio transmitter, receiver, or transceiver) and/or a micropump, and be remotely actuatable via radio signal to compress the reservoir to force the fluid from the reservoir into the balloon tube. In some embodiments, the patient may have a software app on their phone that can send out the radio signal to the VPS system to activate the inflation of the balloon tube 121 from the reservoir 125. In certain embodiments, an active (e.g., electronic) pressure sensor may be disposed in the catheter 105 that senses the pressure in the catheter and, when it senses the pressure in the catheter has dropped either below a predetermined pressure or more than a predetermined amount below a pressure being sensed by another sensor on the outside of the catheter or more abruptly than is normal, any of which could be indicative of an occlusion that justifies activation of the device. For instance, a pressure drop from outside the catheter to inside the catheter of as little as 5 ml is probably indicative of a clogged catheter. In fact, a pressure in the catheter below a predetermined pressure (regardless of the pressure on the outside of the catheter) can be indicative of a clogged catheter. Even further, a sudden drop in pressure can be indicative of a clog.

In yet other embodiments, the balloon tube 121 may be connected via a tube to a self-sealing port near the skin of the patient that the needle of a syringe may be inserted into to inject fluid into the balloon tube to inflate it and then retrieve the fluid back into the syringe. In even further embodiments, surgical intervention may be necessary to inflate the balloon.

In operation, the balloon tube 121 may be inflated for a brief period of time to force out, break up, and/or compress the built up coagula in the pores 107 and/or inner surface of the catheter 105. When inflated, the balloon tube 121 will occlude the entire inner lumen of the catheter 105, thus preventing CFS from flowing through the catheter system as in the normal operation of the system. Accordingly, the period during which the balloon is inflated should be selected to be long enough to achieve the goal of clearing the pores and inner lumen of coagula, but short enough so that it does not further exacerbate the patient's symptoms by restricting the flow of coagula through the catheter 105. In experiments discussed in more detail below, it has been found that 20 seconds of inflation is more than enough to achieve the goals, and 20 seconds of total blockage of the catheter from draining CFS should have little to no physiological impact on the patient.

### Specific Exemplary Embodiment

The invention takes advantage of the large maximum strains⁵³ possible in modern hyperelastic polymers popular in soft robotics devices^{54,55,56} In a preferred embodiment, the inflatable balloon tube 121 should be made of a material having large strain (e.g., greater than 700%, preferably 900% or greater, and more preferably greater than 1000%) so that it consumes as little of the cross-section of the catheter lumen when not in use (i.e., uninflated), yet can be inflated to a size slightly greater than the outer diameter of the catheter without failure. It also should have relatively low hardness (e.g., less that a Shore A hardness of 00-50, and preferably 00-30 or better) so that the portions of the balloon tube adjacent the holes of the catheter body will protrude through the holes when inflated without the need for undue internal pressure. A platinum cured silicone rubber is one preferred material. Ecoflex 00-30 available from Smooth-On, Inc. of Macungie, Pennsylvania is one suitable material. It has a strain (elongation at break) of 900% and a Shore A hardness of 00-30.

FIGS. 2A-2G illustrate one particular exemplary prototype of the system, which is designed for ex vivo testing, and is not representative of how the system would be configured to for deployment in the body (rather, FIG. 1 is representative of how the various components could be configured according to one exemplary in vivo embodiment). Particularly, FIG. 2A is a cut-away plan view of the overall catheter used for testing. FIG. 2B is a blow up view of portion 2B of FIG. 2A. FIG. 2C is a blow up view of portion 2C of FIG. 2A. FIG. 2D shows the proximal end of the catheter system with the inflatable balloon tube uninflated. FIG. 2E shows the proximal end of the catheter system with the inflatable balloon tube inflated. FIG. 2F is a cross-sectional view of the proximal end of the original catheter of the Codman® VP shunt of the prior art that was modified to create the prototype. FIG. 2G is a cross-sectional view of the distal end of the catheter of the prototype catheter (i.e., the modified Codman® catheter).

In this test implementation, the inflating fluid is introduced into the inflatable balloon tube 403 by a syringe (not shown) connected to the rubber tube 405 at the distal port 422 of catheter 405. Any coagula in the test system that enters the lumen of the catheter 405 (through the pores 410) would flow out of the test system via side port 424 of the catheter.

The proximal end (the right end in FIG. 2A) of the lumen of a Codman® VP shunt and the ventricular catheter were modified by increasing the inner diameter of the catheter body 401 from a typical 1.5 mm to 2.77 mm while maintaining the standard outer diameter of 3.0 mm, as shown by comparing FIGS. 2F and 2G. This provided sufficient space to introduce an inflatable silicone tube 403 (formed of Ecoflex 00-30 available from Smooth-On, Macungie, PA USA), with an inner diameter of 1100 μm and an outer diameter of 2340 μm, following the process described by Fan, et al. 57 The Ecoflex 00-30 material was selected due to its ease of fabrication and very large maximum strain of 900%. With this design, the cross-sectional area of the ventricular catheter's lumen was reduced by just 2.3% compared to the original Codman® shunt, producing a negligible effect on the CSF flow characteristics of the catheter 401. The inflatable silicone tube 403 was connected to a 3 mL syringe (BD 3 ml Syringe Luer-Lok™ Tip, available from Becton, Dickinson and Company of Franklin Lakes, New Jersey, USA, not shown) via a relatively rigid, 1.0 mm outer diameter, 0.5 mm inner diameter silicone tubing 405 (Silicone Food Grade Rubber Tube Hose Pipe, available from CGjiogujio of Nanjing, China) for purposes of inflation.

The system was tested using both water and saline as the working fluid for inflating the balloon tube 403, but saline is the working fluid anticipated for use with in in vivo applications. No difference was observed in the system's performance between water and saline.

Bubbles were eliminated from the inner lumen of the silicone tubing 403 by first loading the system with isopropyl alcohol followed by a fluid change to water or saline. The inflated silicone portion inside the shunt rapidly deflated upon release of the pressure on the syringe. The force upon the plunger of the 3 mL syringe necessary to inflate the balloon tube 403 was 1.69 N±0.1 N, which is much less than the approximate 100 N the weakest among males and females from an age of 18 to 85 could generate in past studies.⁵⁸

The inflatable silicone tubing for the balloon tube 403 was fabricated via the following process. A 1.1-mm diameter stainless steel mandrel was first spray-coated with mold release twice, pausing between applications to allow the coating to cure (Mold Release 200, Smooth-On, Inc. Macungie, PA). Then, the Ecoflex 00-30 was mixed and degassed according to standard instructions before immediately being poured upon the mandrel to form a thin, uniform layer that cured at 25° C. for 6 to 8 hours to produce the final product. This tubing was peeled from the mandrel 501 and cut to the desired 25 mm length, corresponding to the length of the portion of the shunt with pores 410. A paraffin mold release was crucial in reducing the risk of tearing the silicone membrane during demolding (i.e., removing the tubing from the mandrel). It was then cleaned using a small amount of isopropyl alcohol (IPA) and then water. Next, the inflatable balloon tube 403 was sealed at one end with a second application of Ecoflex 00-30 to 1.0 mm in length along the inner lumen of the tube. Then, the inflatable tube 403 was attached to the stiffer, non-inflating silicone tubing 405 providing fluid from the syringe using a thin layer of cyanoacrylate “super glue” (CA-MG, available from InfinityBond of Santa Monica, CA) followed by a thin layer of UV epoxy (Norland Optical Adhesive 81, available from Norland Product Inc. of Jamesburg, NJ) and cured with a 365 nm ultraviolet light (365 nm Blacklight UV301D, available from Lightfe of Shenzhen, China).

If one assumes a desired safety factor of 2× and a maximum desired expanded diameter of the balloon tube of 3.3 mm. Using Amax=D/D₀ suggested by Anssari-Benam, et al.⁷⁰, (where Do is the uninflated diameter of the balloon tube and D is the maximum inflated diameter of the balloon tube, the required outer diameter of the silicone during fabrication is 660 μm.

Because the balloon tube 403 will occupy some of the cross-section of the catheter, the thickness, h, of the wall of the balloon tube also should be optimized (e.g., minimized) using

$$h / {h}_{0} = {( {λ}_{θ} {λ}_{z} )}^{- 1} \qquad ( 7 )$$

also suggested by Anssari-Benam⁷⁰, where hₒ is the undeformed thickness of the wall, h is the minimum desired target thickness at maximum inflation (e.g., to keep it from bursting with a safety factor 2×), and λ_{θ} is the stretch (i.e., 1+strain) in the radial direction at maximum inflation, and λ_{z} is the stretch in the axial direction at maximum inflation. Assuming the stretch in the axial direction (z) is equivalent to the stretch in the radial direction with a deformed thickness of h=2.2 mm; using eqn. (7), the undeformed thickness, h₀, should be 6.6 μm. The thinning of the tube wall due to expansion should not be allowed beyond a perceived safe limit. In a preferred embodiment, a safety factor-limited strain of 304% produces a wall thinning of 64%, which is well within the safety margin using a soft material with potential flaws.

### Clogging Agent Model

To test the operation of the system, an occluding agent that is similar to the coagula found in the brain that tends to clog the pores of VP catheters was needed. Several models of occluding agents for ventricular catheters have been presented in the literature.^{50,59-61} A relatively straightforward method is to produce clogging media suitable for testing in an ex vivo model. The approach taken for the tests discussed herein produces clogs in seconds, which is ideal for experiments to test the de-clogging of these shunts. The model of the fluidic system devised for testing included modelling of an increase in intracranial pressure upon clogging of the catheter.

As previously discussed, the CSF composition contains protein and lipids. Eggs are composed of high protein and lipid and have been used in an existing shunt occlusion model with vitelline and chalaza being the primary source of occluding agent⁵⁹. In the model selected for the tests discussed herein, egg albumin, shell membrane, and chalaza were first harvested from the egg. J. Larsen and G. Froning suggest utilizing isopropyl alcohol (IPA) to solidify egg yolk.⁶² The vitelline was first punctured to allow the outflow of egg yolk. Egg yolk and vitelline were separated via 99% IPA. Egg albumin was solidified via heating at T=115-130° C. for about 3 min or until sufficient solidified albumin existed. All the egg components were mixed together to form the occluding agent. This clogging agent formula is used in all the tests involving clogging agents.

### Model of Intracranial Pressure and Installation of Ventricular Catheter

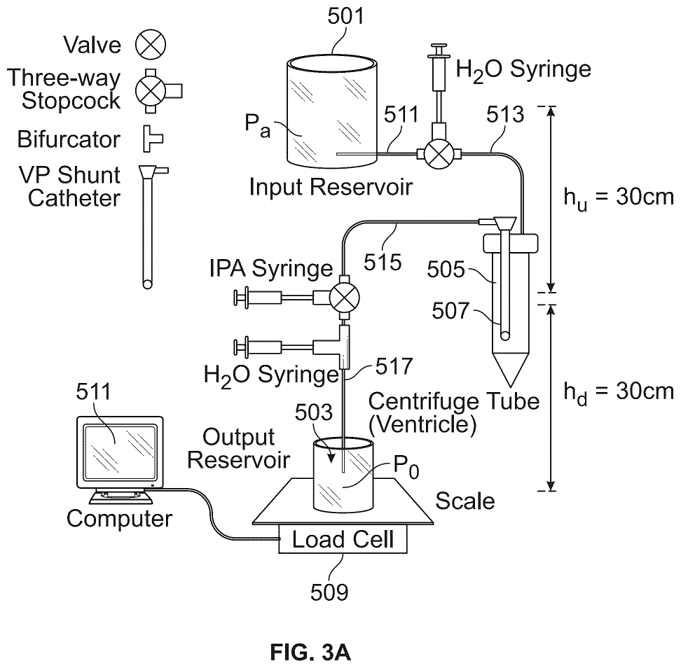

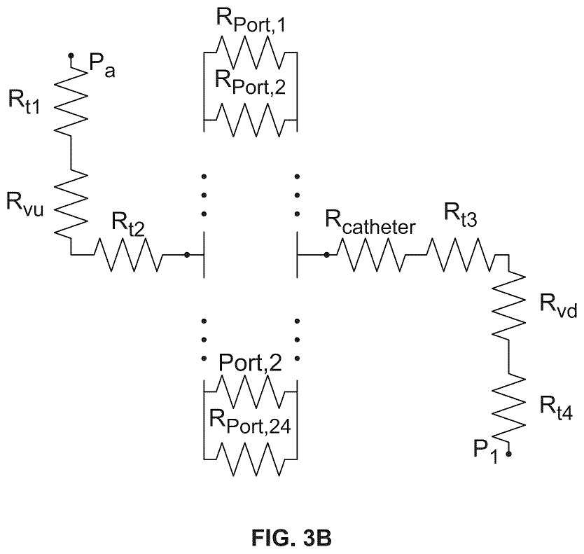

FIG. 3A illustrates a new model for testing the ventricular catheter, intracranial pressure, and CSF flow in which measurements can be taken while the flow circuit is running. In the tests, water was used in place of artificial CSF. Water and CSF have similar dynamic viscosity and density, and, like water, CSF is a Newtonian fluid and is incompressible.¹

A 50 ml Falcon tube 505 served as the ventricle for the tests (hereinafter the ventricle tube). Approximately 3.0 g of the clogging agent was added to the 50 ml ventricle tube before each test trial. Occlusion of the shunt was achieved by vigorous shaking of the ventricle tube after introduction of the clogging agent.

In operation, water flows from the input reservoir 501 to the output reservoir 503 driven by gravity via the ventricle tube 505. The ventricular catheter 507 (substantially as described in connection with FIGS. 2A-2G) is implanted within the ventricle tube 505. The 50 ml volume of the ventricle tube approximates the volume of an adult ventricle.⁵⁹ The appropriate pressures for each portion of the overall system were defined by the relative height of the input reservoir 501, the output reservoir 503, and the ventricle tube 505. The pressure in the input reservoir 501 is denoted herein as pₐ, the pressure in the output reservoir 503 is denoted as pₒ, and the pressure in the ventricle tube 505 is denoted as pᵥₜ. The clogging agent was introduced into the ventricle tube/artificial ventricle 505 before each trial. The output reservoir's 503 mass was measured in 5 s intervals using a simple load cell 509 (HX711, available from Avia Semiconductor of Xiamen, P. R. China) connected to a data collection computer 511 via Arduino. It is possible to determine the pressure in the ventricle tube 505 from knowledge of the component resistances and the flow rate determined from the mass of the output reservoir.

To this end, a simple model of the system was constructed using an electrical circuit analog, as shown in FIG. 3B. The resistance of the silicone tubes 511, 513, 515, 517 connecting the reservoirs 501, 503 and the ventricle tube 505 is denoted by Rₜ₁, Rₜ₂, Rₜ₃, and Rₜ₄, respectively, in FIG. 3B. The resistances of the ventricular catheter 507 ports are R_{port,i} where i={1, 2, 3, . . . , 23, 24}, while R_{catheter} is the resistance from the ventricular catheter's internal lumen. The valves in this experimental system also exhibit unknown fluidic resistance and are represented by Rᵥᵤ and R_{vd} in FIG. 3B. They can be found by calibration with the experiment, as all other component resistances were calculated.

Importantly, the intracranial pressure can be determined. It is defined as pᵥₜ in FIG. 5B. Particularly, using the upstream resistance and the experimental flow rate,

$${p}_{vt} ( {t}_{i} ) = {p}_{a} - Q ( {t}_{i} ) {R}_{u} , \qquad ( 1 )$$

where Rᵤ is the resistance of the upstream portion of the fluidic circuit. The intracranial pressure is the parameter used to evaluate the shunt function. The experimental flow rate Q(tᵢ) at a specific time t was computed using the Euler forward finite difference approximation for the derivative

$$Q ( {t}_{i} ) = \frac{1}{ρΔ t} ( m ( {t}_{i} + Δ t ) - m ( {t}_{i} ) ) \qquad ( 2 )$$

where ρ denotes the density of water and m is the mass of the fluid that passes through a specific location in the system. Hence, the upstream resistance, Rᵤ, can be determined as follows:

$${R}_{u} = {R}_{t 1} + {R}_{t 2} + {R}_{vu} . \qquad ( 3 )$$

The water flow in this circuit may be assumed to be Poiseuille flow,⁶³ producing a flow rate of ^{Q} dependent upon the pressure difference, Δp, from input to output,

$$Q = ( \frac{{A}^{2}}{8 π μ L} ) Δ p , \qquad ( 4 )$$

where A, L, and μ denote the cross-sectional area and length of the tubing and the dynamic viscosity (8.90×10⁴ Pa·s) of the fluid, respectively. Using eqn. (4) and the analogous Ohm's law, Q=Δp/R, each of the resistances in eqn. (3)—except for Rᵥᵤ—are given by

$$R = \frac{8 π μ L}{{A}^{2}} . \qquad ( 5 )$$

The value of Rᵥᵤ is found by comparing the results of the simple analysis model (FIG. 3B) and the experiment (FIG. 3A). The experimental flow rate was slightly slower, and this was assumed to be due to resistance in the valves. Using eqns. (1), (2), and (3), the intracranial pressure, pᵥₜ, at time tᵢ is then given by

$${p}_{vt} ( {t}_{i} ) = γ {h}_{u} - \frac{{R}_{u}}{ρΔ t} ( m ( {t}_{i} + Δ t ) - m ( {t}_{i} ) ) , \qquad ( 6 )$$

where γ is the specific weight (9.807 kN·m⁻³) of water.

### Experimental Results

Injecting saline into the proximal end of the device inflates the silicone balloon tube. When in the catheter, the silicone balloon tube expands and pushes against the interior wall of the catheter body. Those portions of the membrane at the pores protrude out of the pores and appear on the outside of the shunt, as seen in FIG. 2E.

Before the shunt assessment on no clogging compared to clogging, the clogging agent was first prepared according to the protocol described hereinabove. It contains a mixture of all of the egg components. The catheter is clogged in the fluid circuit via vigorous shaking, using the clogging agent prepared from the protocol described earlier.

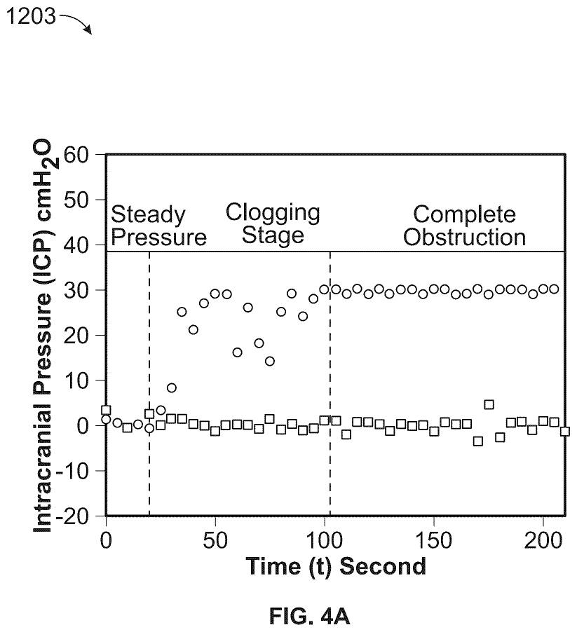

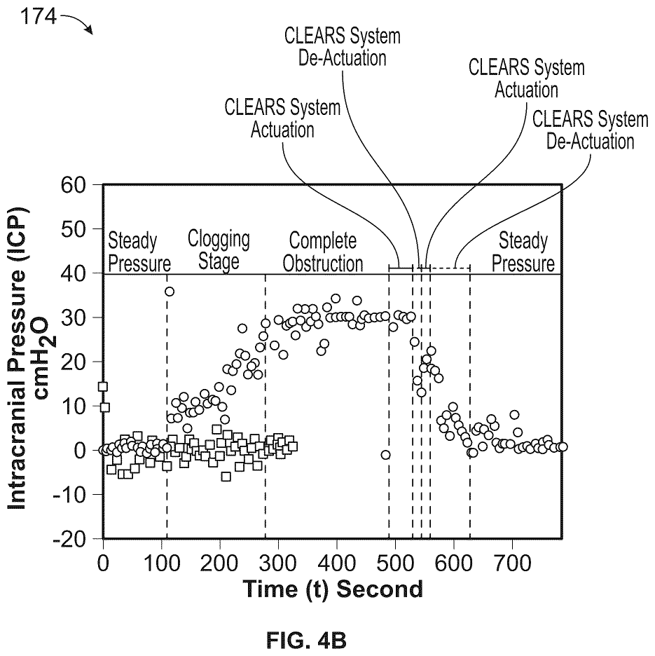

With reference to the graphs of FIGS. 4A and 4B, which plot intracranial pressure as a function of time, four trials were completed, namely, a negative control without a clogging agent (results shown in FIG. 4A by square data points), a positive control with a clogging agent (results shown in FIG. 4A by circular data points), the inventive system without a clogging agent (results shown in FIG. 4B by square data points), and the inventive system with a clogging agent (FIG. 4B by circular data points). The ICP, pᵥₜ, was used to evaluate the relative performance of the inventive system. The square data points in FIG. 4A (the negative control trial) show that the unmodified ventricular catheter from Codman® (the control catheter) has a steady pressure profile of approximately 0 cmH₂O, as expected, since there is no obstruction hindering the flow. The circular data points in FIG. 4A (the positive control trial) show an increase in intracranial pressure after 20 s of no obstruction. The intracranial pressure increases due to the clogging agent blocking the ventricular catheter's pores. At approximately 100 s, ICP rises to 30 cmH₂O and remains at this steady value for the remainder of the trial.

Looking now at FIG. 4B, in can be seen that, in contrast to the Codman® control ventricular catheter, the inventive system resolves the obstruction issues. As shown by the square data points in FIG. 6B (inventive system with no clogging agents), when no clogging agent is introduced, the ICP measured is approximately 0 cmH₂O, as expected. When a clogging agent is introduced (the circular data points in FIG. 4B), the ICP stays at 0 cmH₂O for approximately 105 s, similar to the situation with no clogging agent introduced. During the clogging stage, the centrifuge tube is shaken vigorously to induce obstruction of the pores. The ICP increases as a result of obstruction and complete obstruction occurs when the ICP reaches a steady pressure of 30 cmH₂O, similar to the positive control on the unmodified Codman® ventricular catheter. At/˜485 s, the inventive system was actuated and rapid inflation-deflation of the silicone membrane occurs. Unclogging begins at t˜525 s and the inventive system is de-actuated; resulting in a decrease in ICP values. A continuous relaxation and actuation of the silicone membrane push the clogging agent on the ports away from the shunt. The CLEARS device has a second actuation at t˜545 s and de-actuated at t˜565 s. As a result, the ICP decreases and returns to a steady state of 0 cmH₂O, which is similar to the pressure value before the obstruction is formed on the ports and also similar to the case in which no clogging agent is introduced.

### Additional Embodiments

In the implementation discussed above, the balloon tube inflates uniformly over its axial length. While this type of embodiment provide means to unclog a shunt having the occlusion in the ports or the lumen, it may not unclog obstructions present in other locations of the shunt, the valve, or the tubing from the valve into the peritoneal cavity. However, in other embodiments, the inflatable balloon tube may be provided with variable stiffness over its axial length such that the tube will inflate progressively in the axially direction. For instance, the inflatable tube may be constructed such that the radial stiffness of the tube increases axially from the distal end to the proximal end so that the tube inflates progressively from distal end to proximal end. In one embodiment, such a tube may be fabricated by a gradual increase in thickness of the wall of the tube (e.g., by casting it upside down on the mandrel during fabrication. In such an embodiment, the inflatable tube acts as a sort of plunger that pushes the fluid through the shunt and remaining parts of the system to flush out the obstructions. Moreover, this approach does introduce a working fluid into the brain as a part of the system. Given the strength, durability, and very high maximum strain of Ecoflex and similar polymers, and the very low pressures needed to inflate the balloon tube, the system should be very safe. In fact, overall, the inventive system should present much lower risk to patients than shunts without such unclogging features. However, if the inflatable tube fails, it will introduce the inflating fluid into the brain. Thus, to minimize risk to the patient, in embodiments, the inflating fluid may be a sterilized CSF analog to reduce the risk of injury to the patient should the inflatable balloon tube fail.

While the invention has been described above in connection with ventriculoperitoneal shunts, it should be understood that this is merely one example of many possible applications for the unclogging methods and apparatus disclosed herein. This technology may be used in connection with any pored catheter or other artificial pored lumen that may become clogged. Some such applications include urinary catheters, lumbar peritoneal shunts, PICC (Peripherally Inserted Central Catheter) lines, etc. Also, while the invention has been described mostly in connection with a completely internal VP shunt, it can also be used in connection with short-term VPS in a clinical setting. Short-term VPS in a clinical setting is quite similar to that described above, except that the CFS drains into a container external of the patient's body. Even in the short-term scenario, it is not uncommon for the pores in the catheter to become clogged with coagula.

### CONCLUSION

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer readable medium for execution by a computer or processor. Examples of non-transitory computer-readable storage media include, but are not limited to, a read only memory (ROM), random access memory (RAM), a register, cache memory, semiconductor memory devices, magnetic media such as internal hard disks and removable disks, magneto-optical media, and optical media such as CD-ROM disks, and digital versatile disks (DVDs).

Moreover, in the embodiments described above, processing platforms, computing systems, controllers, and other devices containing processors are noted. These devices may contain at least one Central Processing Unit (“CPU”) and memory. In accordance with the practices of persons skilled in the art of computer programming, reference to acts and symbolic representations of operations or instructions may be performed by the various CPUs and memories. Such acts and operations or instructions may be referred to as being “executed,” “computer executed” or “CPU executed.”

One of ordinary skill in the art will appreciate that the acts and symbolically represented operations or instructions include the manipulation of electrical signals by the CPU. An electrical system represents data bits that can cause a resulting transformation or reduction of the electrical signals and the maintenance of data bits at memory locations in a memory system to thereby reconfigure or otherwise alter the CPU's operation, as well as other processing of signals. The memory locations where data bits are maintained are physical locations that have particular electrical, magnetic, optical, or organic properties corresponding to or representative of the data bits. It should be understood that the exemplary embodiments are not limited to the above-mentioned platforms or CPUs and that other platforms and CPUs may support the provided methods.

The data bits may also be maintained on a computer readable medium including magnetic disks, optical disks, and any other volatile (e.g., Random Access Memory (“RAM”)) or non-volatile (e.g., Read-Only Memory (“ROM”)) mass storage system readable by the CPU. The computer readable medium may include cooperating or interconnected computer readable medium, which exist exclusively on the processing system or are distributed among multiple interconnected processing systems that may be local or remote to the processing system. It is understood that the representative embodiments are not limited to the above-mentioned memories and that other platforms and memories may support the described methods.

In an illustrative embodiment, any of the operations, processes, etc. described herein may be implemented as computer-readable instructions stored on a computer-readable medium. The computer-readable instructions may be executed by a processor of a mobile unit, a network element, and/or any other computing device.

There is little distinction left between hardware and software implementations of aspects of systems. The use of hardware or software is generally (but not always, in that in certain contexts the choice between hardware and software may become significant) a design choice representing cost vs. efficiency tradeoffs. There may be various vehicles by which processes and/or systems and/or other technologies described herein may be effected (e.g., hardware, software, and/or firmware), and the preferred vehicle may vary with the context in which the processes and/or systems and/or other technologies are deployed. For example, if an implementer determines that speed and accuracy are paramount, the implementer may opt for a mainly hardware and/or firmware vehicle. If flexibility is paramount, the implementer may opt for a mainly software implementation. Alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof. Suitable processors include, by way of example, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Application Specific Standard Products (ASSPs); Field Programmable Gate Arrays (FPGAs) circuits, any other type of integrated circuit (IC), quantum computer, and/or a state machine.

The present disclosure is not to be limited in terms of the particular embodiments described in this application, which are intended as illustrations of various aspects. Many modifications and variations may be made without departing from its spirit and scope, as will be apparent to those skilled in the art. No element, act, or instruction used in the description of the present application should be construed as critical or essential to the invention unless explicitly provided as such. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods or systems.

It is also to be understood that the terminology used herein is for the purpose of describing particular embodiments only, and is not intended to be limiting.

In certain representative embodiments, several portions of the subject matter described herein may be implemented via Application Specific Integrated Circuits (ASICs), Field Programmable Gate Arrays (FPGAs), digital signal processors (DSPs), and/or other integrated formats. However, those skilled in the art will recognize that some aspects of the embodiments disclosed herein, in whole or in part, may be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein may be distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc., and a transmission type medium such as a digital and/or an analog communication medium (e.g., a fiber optic cable, a waveguide, a wired communications link, a wireless communication link, etc.).

The herein described subject matter sometimes illustrates different components contained within, or connected with, different other components. It is to be understood that such depicted architectures are merely examples, and that in fact many other architectures may be implemented which achieve the same functionality. In a conceptual sense, any arrangement of components to achieve the same functionality is effectively “associated” such that the desired functionality may be achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, where only one item is intended, the term “single” or similar language may be used. As an aid to understanding, the following appended claims and/or the descriptions herein may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”). The same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, the terms “any of” followed by a listing of a plurality of items and/or a plurality of categories of items, as used herein, are intended to include “any of,” “any combination of,” “any multiple of,” and/or “any combination of multiples of” the items and/or the categories of items, individually or in conjunction with other items and/or other categories of items. Moreover, as used herein, the term “set” or “group” is intended to include any number of items, including zero. Additionally, as used herein, the term “number” is intended to include any number, including zero.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” “greater than,” “less than,” and the like includes the number recited and refers to ranges which can be subsequently broken down into subranges as discussed above. Finally, as will be understood by one skilled in the art, a range includes each individual member. Thus, for example, a group having 1-3 items refers to groups having 1, 2, or 3 items. Similarly, a group having 1-5 items refers to groups having 1, 2, 3, 4, or 5 items, and so forth.

Moreover, the claims should not be read as limited to the provided order or elements unless stated to that effect. In addition, use of the terms “means for” in any claim is intended to invoke 35 U.S.C. § 112, ¶6 or means-plus-function claim format, and any claim without the terms “means for” is not so intended.

Although the invention is illustrated and described herein with reference to specific embodiments, the invention is not intended to be limited to the details shown. Rather, various modifications may be made in the details within the scope and range of equivalents of the claims and without departing from the invention.

Throughout the disclosure, one of skill understands that certain representative embodiments may be used in the alternative or in combination with other representative embodiments.

### REFERENCES

a) Medically Advanced Devices Laboratory, Center for Medical Devices and Biomechanics, Department of Mechanical and Aerospace Engineering, Jacobs School of Engineering and Department of Surgery, School of Medicine, University of California San Diego, La Jolla, CA 92093-0411 USA
b) https://friend.ucsd.edu; Corresponding author: jfriend@ucsd.edu
[1] H. Balasundaram, S. Sathyamoorthi, U. Fernandez-Gamiz, S. Noeiaghdam, and S. S. Santra, “Hydrocephalic cerebrospinal fluid flowing rotationally with pulsatile boundaries: a mathematical simulation of the thermodynamical approach,” Theoretical and Applied Mechanics Letters 13, 100418 (2023).
[2] J. M. Rubino and J. P. Hogg, “Neuroanatomy, cerebral aqueduct (sylvian),” (2019).
[3] R. M. Fame, C. Cortes-Campos, and H. L. Sive, “Brain ventricular system and cerebrospinal fluid development and function: light at the end of the tube: a primer with latest insights,” BioEssays 42, 1900186 (2020).
[4] A. Toma, “Hydrocephalus in adults,” in Principles of Neurological Surgery (Elsevier, 2018) pp. 822-831.
[5] L. N. Telano and S. Baker, “Physiology, cerebral spinal fluid,” (2018).
[6] P. Kompanfkova and V. Bryja, “Regulation of choroid plexus development and its functions,” Cellular and Molecular Life Sciences 79, 304 (2022).
[7] M. Ueno, Y. Chiba, R. Murakami, Y. Miyai, K. Matsumoto, K. Wakamatsu, T. Nakagawa, G. Takebayashi, N. Uemura, K. Yanase, et al., “Transporters, ion channels, and junctional proteins in choroid plexus epithelial cells,” Biomedicines 12, 708 (2024).
[8] C. Municio, L. Carrero, D. Antequera, and E. Carro, “Choroid plexus aquaporins in csf homeostasis and the glymphatic system: Their relevance for alzheimer's disease,” International Journal of Molecular Sciences 24, 878 (2023).
[9] H. Reiber, “Flow rate of cerebrospinal fluid (csf)—a concept common to normal blood-csf barrier function and to dysfunction in neurological diseases,” Journal of the neurological sciences 122, 189-203 (1994).
[10] R. Spector, S. R. Snodgrass, and C. E. Johanson, “A balanced view of the cerebrospinal fluid composition and functions: Focus on adult humans,” Experimental neurology 273, 57-68 (2015).
[11] L. Diotalevi, J.-M. Mac-Thiong, and Y. Petit, “Cerebrospinal fluid model formulation affects global and local behaviour of the spinal cord submitted to transverse traumatic compression,” in International Symposium on Computer Methods in Biomechanics and Biomedical Engineering (Springer, 2023) pp. 113-120.
[12] R. Kamel, H. Elbosraty, M. Hafez, and T. Kandil, “Physiology of csf,” in CSF Rhinorrhea: Pathophysiology, Diagnosis and Skull Base Reconstruction (Springer, 2022) pp. 15-20.
[13] F. B. Shipley, N. Dani, H. Xu, C. Deister, J. Cui, J. P. Head, C. Sadegh, R. M. Fame, M. L. Shannon, V. I. Flores, et al., “Tracking calcium dynamics and immune surveillance at the choroid plexus blood-cerebrospinal fluid interface,” Neuron 108, 623-639 (2020).
[14] J. Xie, A. Bruggeman, C. De Nolf, C. Vandendriessche, G. Van Imschoot, E. Van Wonterghem, L. Vereecke, and R. E. Vandenbroucke, “Gut microbiota regulates blood-cerebrospinal fluid barrier function and a/3 pathology,” The EMBO Journal 42, el 11515 (2023).
[15] 0. Cousins, A. Hodges, J. Schubert, M. Veronese, F. Turkheimer, J. Miyan, B. Engelhardt, and F. Roncaroli, “The blood-csf-brain route of neurological disease: The indirect pathway into the brain,” Neuropathology and applied neurobiology 48, e12789 (2022).
[16] K. Saito, K. Hattori, S. Hidese, D. Sasayarna, T. Miyakawa, R. Matsumura, M. Tatsumi, Y. Yokota, M. Ota, H. Hori, et al., “Profiling of cerebrospinal fluid lipids and their relationship with plasma lipids in healthy humans,” Metabolites 11, 268 (2021).
[17] R. Khazim, Z. Dannawi, K. Spacey, M. Khazim, S. Lennon, A. Reda, and A. Zaidan, “Incidence and treatment of delayed symptoms of csf leak following lumbar spinal surgery,” European Spine Journal 24, 2069-2076 (2015).
[18] D. Satzer and D. J. Guillaume, “Hearing loss in hydrocephalus: a review, with focus on mechanisms,” Neurosurgical review 39, 13-25 (2016).
[19] T. Y. Kim, G. Stewart, M. Voth, J. A. Moynihan, and L. Brown, “Signs and symptoms of cerebrospinal fluid shunt malfunction in the pediatric emergency department,” Pediatric emergency care 22, 28-34 (2006).
[20] 0. Sacko, S. Boetto, V. Lauwers-Cances, M. Dupuy, and F.-E. Roux, “Endoscopic third ventriculostomy: outcome analysis in 368 procedures,” Journal of Neurosurgery: Pediatrics 5, 68-74 (2010).
[21] Y. Bu, M. Chen, T. Gao, X. Wang, X. Li, and F. Gao, “Mechanisms of hydrocephalus after intraventricular haemorrhage in adults,” Stroke and Vascular Neurology 1, 23 (2016).
[22] S. P. Emery, S. Greene, M. Elsisy, K. Chung, S.-H. Ye, S. Kim, W. R. Wagner, N. Hazen, and Y. Chun, “In vitro and in vivo assessment of a novel ultra-flexible ventriculoamniotic shunt for treating fetal hydrocephalus,” Journal of Biomaterials Applications 37, 1423-1435 (2023).
[23] R. J. Edwards, S. M. Dombrowski, M. G. Luciano, and I. K. Pople, “Chronic hydrocephalus in adults,” Brain pathology 14, 325-336 (2004).
[24] M. Juhler, Hydrocephalus (Springer, 2020).
[25] L. Szefczyk-Polowczyk and M. Mandera, “Functioning of the children with hydrocephalus,” Acta Neurologica Belgica 120, 345-353 (2020).
[26] R. J. Schain, “Carbonic anhydrase inhibitors in chronic infantile hydrocephalus,” American Journal of Diseases of Children 117, 621-625 (1969).
[27] G. Aimard, A. Vighetto, J. Gabet, P. Bret, and E. Henry, “Acetazolamide: an alternative to shunting in normal pressure hydrocephalus? preliminary results,” Revue neurologique 146, 437-439 (1990).
[28] C. P. Bondurant and D. F. Jimenez, “Epidemiology of cerebrospinal fluid shunting,” Pediatric neurosurgery 23, 254-259 (1995).
[29] Y. Serarslan, A. Yilmaz, M. C::akir, E. Gtizel, A. Akakin, A. Gtizel, B. Urfali, M. Aras, M. E. Kaya, and N. Yilmaz, “Use of programmable versus nonprogrammable shunts in the management of normal pressure hydrocephalus: A multicenter retrospective study with cost-benefit analysis in turkey,” Medicine 96, e8185 (2017).
[30] L. Momani, A. R. Alkharabsheh, and W. Al-Nuaimy, “Design of an intelligent and personalized shunting system for hydrocephalus,” in 2008 30th Annual International Conference of the IEEE Engineering in Medicine and Biology Society (IEEE, 2008) pp. 779-782.
[31] 0. J. Soler, M. Bao, D. Jaiswal, H. P. Zaveri, M. L. DiLuna, R. A. Grant, and K. Hoshino, “Focus: medical technology: a review of cerebral shunts, current technologies, and future endeavors,” The Yale Journal of Biology and Medicine 91,313 (2018).
[32] P. Sokal, M. Birski, M. Rusinek, D. Paczkowski, P. Zielinski, and A. Harat, “Endoscopic third ventriculostomy in treatment of hydrocephalus,” Videosurgery and Other Miniinvasive Techniques 7, 280-285 (2012).
[33] A. Sarmast, N. Khursheed, A. Ramzan, F. Shaheen, A. Wani, S. Singh, Z. Ali, and B. Dar, “Endoscopic third ventriculostomy in noncommunicating hydrocephalus: report on a short series of 53 children,” Asian Journal of Neurosurgery 14, 35-40 (2019).
[34] E. Brichtova, M. Chlachula, T. Hrbac, R. Lipina, et al., “Endoscopic third ventriculostomy in previously shunted children,” Minimally Invasive Surgery 2013 (2013).
[35] Y. R. Yadav, V. Parihar, S. Pantle, H. Namdev, and M. Agarwal, “Endoscopic third ventriculostomy,” Journal of neurosciences in rural practice 3, 163-173 (2012).
[36] W. J. Hader, R. L. Walker, S. T. Myles, and M. Hamilton, “Complications of endoscopic third ventriculostomy in previously shunted patients,” Operative Neurosurgery 63, ONS168-ONS175 (2008).
[37] V. Levrini, Z. Czosnyka, I. Lawes, A. G. Kolias, and R. Mannion, “Shunt testing in vivo: Illustration of partially obstructed ventricular catheter by in-growing choroid plexus,” Cureus 12 (2020).
[38] N. Mansoor, 0. Solheim, 0. A. Fredriksli, and S. Gulati, “Revision and complication rates in adult shunt surgery: a single-institution study,” Acta Neurochirurgica 163, 447-454 (2021).
[39] R. V. Patwardhan and A. Nanda, “Implanted ventricular shunts in the united states: the billion-dollar-a-year cost of hydrocephalus treatment,” Neurosurgery 56, 139-145 (2005).
[40] E. Nabbanja, J. D. Pickard, A. D. Lalou, and Z. H. Czosnyka, “Use of csf infusion studies to unblock occluded hydrocephalus ventricular shunt catheters: a preliminary report of two patients,” Case Reports 2018, bcr-2017 (2018).
[41] N. Gupta, J. Park, C. Solomon, D. A. Kranz, M. Wrensch, and Y. W. Wu, “Long-term outcomes in patients with treated childhood hydrocephalus,” Journal of Neurosurgery: Pediatrics 106, 334-339 (2007).
[42] M. Choux, L. Genitori, D. Lang, and G. Lena, “Shunt implantation: reducing the incidence of shunt infection,” Journal of neurosurgery 77, 875-880 (1992).
[43] P. Kofoed Mansson, S. Johansson, M. Ziebell, and M. Juhler, “Forty years of shunt surgery at Rigshospitalet, Denmark: a retrospective study comparing past and present rates and causes of revision and infection,” BMJ Open 7 (2017), 10.1136/bmjopen-2016-013389.
[44] W. Suryaningtyas, I. A. R. Ranuh, and M. A. Parenrengi, “Shunt exposure as a ventriculoperitoneal shunt complication: A case series,” International Journal of Surgery Case Reports 79, 484-491 (2021).
[45] M. A. Samuels, S. Amin-Hanjani, W. F. Pirl, S. K. Feske, and P. M. Black, “Chapter 16-hydrocephalus and disorders of cerebrospinal fluid flow,”, 159-167 (2003).
[46] M. Perras, N. McCauley, T. Stead, L. Ganti, and B. Desai, “Ventriculoperitoneal shunts in the emergency department: A review.” Cureus 12, e6857 (2020).
[47] H. B. Gilbert and R. J. Webster, “Rapid, reliable shape setting of superelastic nitinol for prototyping robots,” IEEE robotics and automation letters 1, 98-105 (2015).
[48] G. K. Nandasiri, A. Ianakiev, and T. Dias, “Hyperelastic properties of platinum cured silicones and its applications in active compression,” Polymers 12, 148 (2020).
[49] M. Galarza, A. Gimenez, J. M. Amigo, M. Schuhmann, R. Gazzeri, U. Thomale, and J. P. McAllister, “Next generation of ventricular catheters for hydrocephalus based on parametric designs,” Child's Nervous System 34, 267-276 (2018).
[50] A. Patel, D. Qi, J. Boyle, M. Morris, and J. Lin, “Dual catheter and double-lumen cerebrospinal fluid shunt systems with backflow mechanisms,” Child's Nervous System 40, 135-143 (2024).
[51] B. R. Lutz, P. Venkataraman, and S. R. Browd, “New and improved ways to treat hydrocephalus: pursuit of a smart shunt,” Surgical neurology international 4, S38 (2013).
[52] 1. Joanny and P.-G. De Gennes, “Upward creep of a wetting fluid: a scaling analysis,” Journal de Physique 47, 121-127 (1986).
[53] T. Gopesh and J. Friend, “Facile analytical extraction of the hyperelastic constants for the two-parameter Mooney-Rivlin model from experiments on soft polymers,” Soft Robotics 8, 365-370 (2020).
[54] K. Suzumori, S. Iikura, and H. Tanaka, “Flexible microactuator for miniature robots,” in Micro Electro Mechanical Systems, 1991, MEMS′91, Proceedings. An Investigation of Micro Structures, Sensors, Actuators, Machines and Robots. IEEE (IEEE, 1991) pp. 204-209.
[55] K. Suzumori, A. Koga, F. Kondo, and R. Haneda, “Integrated flexible microactuator systems,” Robotica 14, 493-498 (1996).
[56] T. Gopesh, J. H. Wen, D. Santiago-Dieppa, B. Yan, J. S. Pannell, A. Khalessi, A. Norbash, and J. Friend, “Soft robotic steerable microcatheter for the endovascular treatment of cerebral disorders,” Science robotics 6, eabf0601 (2021).
[57] D. Fan, Y. Liao, W. Wu, P. Zhang, X. Yang, R. Zhu, Y. Wang, C. Yang, and H. Wang, “Flow casting soft shells with geometrical complexity and multifunctionality,” Advanced Materials Technologies 8, 2201640 (2023).
[58] Y.-C. Wang, R. W. Bohannon, X. Li, B. Sindhu, and J. Kapellusch, “Hand-grip strength: Normative reference values and equations for individuals 18 to 85 years of age residing in the united states,” Journal of Orthopaedic & Sports Physical Therapy 48, 685-693 (2018).
[59] D. Qi, A. Patel, R. Dunwoody, S. McCall, S. Bach, and J. Lin, “Proximal ventricular shunt catheter occlusion model,” Child's Nervous System 39, 205-210 (2023).
[60] F. Fliirenbrock, L. Rosalia, A. Podgor, K. Sapozhnikov, N. E. Trimmel, M. Weisskopf, M. F. Oertel, E. Roche, M. N. Zeilinger, L. Korn, et al., “A soft robotic actuator system for in vivo modeling of normal pressure hydrocephalus,” IEEE Transactions on Biomedical Engineering (2023).
[61] E. Olson, J. Garst, J. Blank, H. Abbott, A. Shaffer, Z. Anderson, K. Nair, and J. Lin, “Simulation of proximal catheter occlusion and design of a shunt tap aspiration system,” Child's Nervous System 37, 895-901 (2021).
[62] J. Larsen and G. Froning, “Extraction and processing of various components from egg yolk,” Poultry Science 60, 160-167 (1981).
[63] B. Kirby, Micro- and Nanoscale Fluid Mechanics: Transport in Microfluidic Devices (Cambridge University Press, London, U. K., 2013).
[64] F. Javeed, A. Mohan, U. U. Wara, L. Rehman, and M. Khan, “Ventriculoperitoneal shunt surgery for hydrocephalus: one of the common neurosurgical procedures and its related problems,” Cureus 15 (2023).
[65] M. Ferras, N. McCauley, T. Stead, L. Ganti, and B. Desai, “Ventriculoperitoneal shunts in the emergency department: a review,” Cureus 12 (2020).
[66] H. J. Garton, J. R. Kestle, and J. M. Drake, “Predicting shunt failure on the basis of clinical symptoms and signs in children,” Journal of neurosurgery 94, 202-210 (2001).
[67] S. H. Weisenberg, S. C. TerMaath, C. E. Seaver, and J. A. Killeffer, “Ventricular catheter development: past, present, and future,” Journal of neurosurgery 125, 1504-1512 (2016).
[68] S. C. Lee and C. J. Lueck, “Cerebrospinal fluid pressure in adults,” Journal of Neuroophthalmology 34, 278-283 (2014).
[69] R. C. Reis, R. H. G. Yamashita, D. J. F. Solla, L. F. Ramin, M. J. Teixeira, and F. C. G. Pinto, “Treatment of idiopathic normal pressure hydrocephalus with a novel programmable valve: Prospective evaluation of costs, efficacy, and safety,” Asian Journal of Neurosurgery 17 (2023).
[70] A. Anssari-Benam, A. Bucchi, and G. Saccomandi, “Modelling the inflation and elastic instabilities of rubber-like spherical and cylindrical shells using a new generalised neo-hookean strain energy function,” Journal of Elasticity 151, 15-45 (2022).
[71] Stephen Kralik, Nilesh Desai, Avner Meoded, Thierry A. G. M. Huisman, Fetal, Neonatal and Pediatric Neuroradiology, Elsevier, May 16, 2023, Paperback ISBN: 9780323796958, eBook ISBN: 9780323796965

What is claimed:

1. A ventriculoperitoneal (VP) shunt system comprising:
a catheter for placement in a ventricle, the catheter comprising a tube, the tube having an outer diameter and an inner diameter, and defining an inner lumen, the catheter tube having a plurality of holes in a wall thereof to allow infiltration of fluids from external of the catheter into the inner lumen via the holes;
a drainage tube in fluid communication with the inner lumen of the catheter for draining fluid that infiltrates the catheter; and
an inflatable tube disposed within the inner lumen of the catheter, the inflatable tube having a diameter smaller than a diameter of the inner lumen of the catheter when the inflatable tube is uninflated and inflatable to a diameter greater than the outer diameter of the catheter.

2. The VP shunt of claim 1 wherein the inflatable tube is formed of a material having a strain of no less than about 900%.

3. The VP shunt of claim 2 wherein the inflatable tube is formed of an elastic material having a hardness of Shore A 20 or less.

4. The VP shunt of claim 1 wherein the inflatable tube is formed of Ecoflex 00-30.

5. The VP shunt system of claim 1 further comprising:
means for inflating the inflatable tube to an unrestricted diameter greater than the outer diameter of the catheter tube.

6. The VP shunt of claim 1 further comprising:
a reservoir for containing a fluid for inflating the balloon tube; and
a connecting tube in fluid communication with the reservoir and the balloon tube.

7. The VP shunt of claim 6 further comprising a pump configured to compress the reservoir upon activation.

8. The VP shunt of claim 7 further comprising a radio receiver configured to receive a particular signal and to activate the pump responsive to receipt of the particular signal.

9. The method of claim 6 wherein the reservoir is implantable adjacent the skin of a patient, whereby the reservoir can be compressed to force fluid in the reservoir from the reservoir into the balloon tube via the connecting tube by pressing on the skin of the patient adjacent the reservoir.

10. The VP shunt of claim 6 wherein the reservoir is a syringe and the connecting tube is a needle.

11. The VP shunt of claim 1 wherein the balloon tube is coaxial with the catheter tube.

12. The VP shunt of claim 1 further comprising a pressure sensor disposed within the inner lumen, the pressure sensor configured to detect when the pressure in the inner lumen drops below a predetermined pressure.

13. The VP shunt of claim 12 further comprising:
a radio transmitter connected to the pressure sensor and configured to transmit a radio signal when the sensor detects that the pressure in the inner lumen has dropped below the predetermined pressure.

14. A method of unclogging an in vivo artificial tube having an outer diameter and an inner diameter, and defining an inner lumen, the tube having a plurality of holes in a wall thereof to allow infiltration of fluids from external of the tube into the inner lumen via the holes, the method comprising:
disposing an inflatable tube within the inner lumen of the artificial tube, the inflatable tube having a diameter smaller than a diameter of the inner lumen when the inflatable tube is uninflated and inflatable to a diameter greater than the outer diameter of the artificial tube;
detecting a condition indicative of clogging of the artificial tube; and
inflating the inflatable tube sufficiently to contact the wall of the artificial lumen and protrude through the holes.

15. The method of claim 14 wherein inflating the inflatable tube comprises:
engaging a syringe containing sufficient fluid to inflate the inflatable tube in fluid communication with the inflatable tube; and
forcing the fluid from the syringe into the inflatable tube.

16. The method of claim 14 wherein the detecting comprises detecting that the pressure in the lumen of the artificial tube below a predetermined threshold.

17. The method of claim 14 wherein the detecting comprises detecting clinical symptoms of hydrocephalus.

18. The method of claim 14 wherein inflating the inflatable tube comprises compressing a fluid-filled reservoir that is in fluid communication with the inflatable tube.

19. The method of claim 18 wherein compressing the reservoir comprises activating a pump that compresses the reservoir.

20. The method of claim 18 wherein the reservoir is implanted adjacent the skin of a patient and wherein compressing the reservoir comprises pressing on the skin of the patient adjacent the reservoir.
